(19) United States
(12) Patent Application Publication
(10) Pub. No.: US 2025/0339668 A1
(43) Pub. Date: Nov. 6, 2025

(54) VASCULAR PUMP

(71) Applicant: Second Heart Assist, Inc., Salt Lake City, UT (US)

(72) Inventor: Howard J. Leonhardt, Mission Viejo, CA (US)

(21) Appl. No.: 18/869,615

(22) PCT Filed: Jun. 2, 2023
(86) PCT No.: PCT/US2023/024232

§ 371 Date: Nov. 26, 2024

Related U.S. Application Data
(60) Provisional application No. 63/348,473, filed on Jun. 2, 2022.

(51) Int. Cl. A61M 60/816; A61M 60/135; A61M 60/216; A61M 60/508; A61M 60/808

(52) U.S. Cl. CPC A61M 60/816; A61M 60/135; A61M 60/216; A61M 60/508; A61M 60/808; A61M 2205/0294; A61M 2205/04; A61M 2205/3317; A61M 2205/3327; A61M 2205/3331; A61M 2205/3561; A61M 2205/3592; A61M 2210/12

(57) ABSTRACT

Described is a device that includes a distal end and a proximal end, the device having a sensor attached at the distal end for receiving electromagnetic frequencies for controlling an impeller. The impeller moves fluid within a conduit based upon the received electromagnetic frequencies. Two or more chambers are positioned proximally and distally relative to the impeller. The impeller is positioned between the two or more chambers. The chambers preferably contain vibrational or piezoelectric materials to move or vibrate upon receiving one or more of the electromagnetic frequencies so as to reduce thrombosis.

### CROSS-REFERENCE TO RELATED APPLICATIONS

This application is a national phase entry of International Patent Application PCT/US2023/024232, filed Jun. 2, 2023, designating the United States of America and published in English as International Patent Publication WO2023/235537 A1 on Dec. 7, 2023, which claims the benefit of the filing date of U.S. Provisional Patent Application Ser. No. 63/348,473, filed Jun. 2, 2022, for “VASCULAR PUMP.”

### TECHNICAL FIELD

The application relates generally to devices useful for medical treatment, and more particularly to a magnetically and/or electromagnetically activated circulatory assist device(s) that may be placed within a blood vessel (e.g., an artery or vein) and related methods. More particularly, the application discloses intravascular circulatory assist devices that include pulsating balloons or chambers that include material that is configured to vibrate, resonate, or move in response to an applied electrical or magnetic field, and related methods of using the circulatory assist devices.

### BACKGROUND

Maintaining and improving blood flow before, during, or after medical procedures, heart conditions, and heart failure may involve percutaneous coronary intervention (“PCI”). PCI may be vital to preventing or eliminating the negative effects of deep vein thrombosis (DVT), pulmonary embolism, and venous thromboembolism (VTE). Circulatory assist devices, such as circulatory assist pumps, left ventricle assist devices, pacemakers, and long-term use catheters, are often used in PCI to reduce, prevent, or eliminate angina, blood clots, calcification, plaque buildup (e.g., atherosclerosis), and/or plaque formation on blood contact surfaces.

Hemolysis is a condition in which red blood cells are broken down or damaged. Circulatory assist devices that use impellers may facilitate or cause hemolysis. Internally, circulatory assist devices may have blood clots formed therein, which may be dislodged and transferred to the body, causing the negative effects these devices are intended to prevent or eliminate.

U.S. Pat. No. 8,617,239 to Reitan (Dec. 13, 2013), the contents of which are incorporated herein by this reference, relates to a catheter pump to be positioned in the ascending aorta near the aortic valve of a human being, comprising an elongated sleeve with a drive cable extending through the sleeve and connectable at its proximal end to an external drive source and a drive rotor near the distal end of the drive cable mounted on a drive shaft being connected with the drive cable. The drive rotor consists of a propeller enclosed in a cage and the propeller and the cage are foldable from an insertion position close to the drive shaft to an expanded working position, which are characterized by means for anchoring the drive rotor in the ascending aorta near the aortic valve after insertion. Also described is a method to position the pump of a catheter pump in the ascending aorta just above the aortic valve.

U.S. Pat. No. 8,617,239 to Reitan builds upon an earlier patent of Reitan, i.e., U.S. Pat. No. 5,749,855 to Reitan (May 12, 1998), the contents of which are also incorporated herein by this reference, which relates to a drive cable, with one end of the drive cable being connectable to a drive source and a collapsible drive propeller at the other end of the drive cable. The collapsible drive propeller is adjustable between a closed configuration in which the collapsible drive propeller is collapsed on the drive cable and an open configuration in which the collapsible drive propeller is expanded so as to be operative as an impeller. A sleeve extends between one side of the collapsible drive propeller and the other side of the collapsible drive propeller with the sleeve being movable between configurations in which the collapsible drive propeller is in the open and closed configuration. A lattice cage is arranged surrounding the propeller and is folded out at the same time as the propeller.

An even earlier blood pumping catheter is described in U.S. Pat. No. 4,753,221 to Kensey et al. (Jun. 28, 1988), the contents of which are incorporated herein by this reference. Kensey et al. relates to an elongated catheter for pumping blood through at least a portion of a subject's vascular system. The catheter is of a sufficiently small diameter and flexibility to enable it to be passed through the vascular system so that the distal end portion of the catheter is located within or adjacent the patient's heart. A rotatable pump is located at the distal end of the catheter and is rotated by drive means in the catheter. The distal end portion of the catheter includes an inlet for blood to flow therein and an outlet for blood to flow therefrom. The catheter is arranged so that blood is pumped by the catheter's pump through the heart and into the vascular system without requiring any pumping action of the heart.

Other catheter pumps are known from US 2008/0132748 A1, US 2008/0114339 A1, U.S. Pat. No. 11,602,627, and WO03/103745A2, the contents of each of which are incorporated herein by this reference.

The above-described background relating to circulatory assist devices is merely intended to provide a contextual overview of some current issues and is not intended to be exhaustive. Other contextual information may become apparent to those of ordinary skill in the art upon review of the following description, which includes example embodiments.

### BRIEF SUMMARY

Described herein is a vascular device that simulates pulsatility of a patient's blood flow by utilizing at least one electromagnetically and/or magnetically activated chamber (e.g., balloon). Embodiments herein may be used to facilitate blood flow within the subject's blood vessel(s). In addition, embodiments hereof may be utilized to compress and/or break up plaque that has accumulated within the subject's blood vessel(s) to improve flow through the respective blood vessel(s).

In some embodiments, such a device includes a distal end and a proximal end, having a sensor attached at the distal end for receiving electromagnetic frequencies for controlling an impeller. The impeller moves intravenous fluid based upon the received electromagnetic frequencies. Two or more (e.g., balloon) chambers are positioned proximally and distally relative to the impeller. The impeller is positioned between the two or more chambers. The chambers may contain vibrational or piezoelectric materials to move or vibrate upon receiving one or more of the electromagnetic frequencies.

In some embodiments, the circulatory assist device utilizes one or more devices (e.g., electromagnets) positioned just above the skin of the patient that generate an electric, magnetic, and/or electromagnetic field via transcutaneous transmission to partially actuate and/or fully actuate one or more actuatable portion(s) of the device (e.g., impeller, balloon, etc.). In other words, the electric, magnetic, or electromagnetic field transfers through the subject's skin, tissue, and blood vessel(s) to the device within the patient's blood vessel. In response to the applied electric, magnetic, or electromagnetic field, one or more actuatable portions of the device within the patient's blood vessel may move (e.g., vibrate, actuate, constrict, or expand).

Diametric movements (e.g., constriction and/or expansion) of the actuatable portion(s) of the device contributes to pulsating blood flow within the blood vessel. In addition, constriction and/or expansion of the actuatable portion(s) (e.g., balloons) of the device may be coordinated with the pulsating blood flow originating from the patient's heart.

The electric, magnetic, or electromagnetic field may be applied to reduce thrombosis associated with the device in various ways. For example, in some embodiments, the magnetic field may be applied by a single electromagnet external to the subject. In embodiments with a single electromagnet, the magnetic field may be modulated and/or concentrated (e.g., focused, localized) in a plane and/or at one of the actuatable portions of the balloon or sensor. In addition, a concentrated magnetic field may be steered as desired, such as through the use of a controller that directs the magnetic field.

In additional embodiments, the magnetic field may be applied by multiple selectively actuatable external electromagnets. The electromagnets may be arranged in series so that the electromagnets can be activated then deactivated in succession to change the vibration of (e.g., pulsate, constrict, or expand) portions of the device.

In further embodiments, the magnetic field may be applied by one or more internal electromagnets that are positioned within the device that is positioned within the subject's blood vessel(s). For example, the device may generate an electrical current within a distally located sensor that activates internal electromagnets to generate an internal magnetic field.

In some embodiments, the device includes a drive shaft with multiple independently actuatable portions (e.g., balloons or chambers) arranged in series along a length of the drive shaft. The independently actuatable portions or sections of actuatable portions of the device are each individually configured to change movement (e.g., vibration frequency, constrict, or expand) in response to an applied magnetic field. For example, each actuatable portion or section of each actuatable portion of the device may be independently actuated by a concentrated (e.g., focused, localized) magnetic field applied to the respective actuatable portion, which may change the vibration frequency of the respective actuatable portion of the device. In some embodiments, each actuatable portion or each section of each actuatable portion of the device may be successively actuated (e.g., by a moving magnetic field) such that the vibration of the balloons successively changes along the length of the device so as to reduce thrombosis associated with a placed such device.

Thus described, among other things, is a pulsating drive shaft that includes one or more balloons. A first balloon or chamber of the device may be biased to vibrate at a first frequency. A second balloon or chamber of the device may be biased to vibrate at a second frequency such that at least a portion of device is configured to simulate blood flow of a blood vessel of a patient in response to an applied magnetic field.

In some embodiments, a device may include additional components beyond the drive shaft and balloons. For example, the device may include one or more electromagnets that may remain external to the subject, and that generate a magnetic field to change the movement of the actuatable portion(s) of the device.

Furthermore, the device may include (or be associated with) a controller and/or a power source in electronic communication with the electromagnet(s). The controller may be configured to modulate the electric, electromagnetic, or magnetic field (e.g., of the electromagnet) and/or may even be configured to steer the electric, electromagnetic, or magnetic field. In embodiments that include multiple electromagnets, the controller may be configured to selectively activate and deactivate each electromagnet to move the location of the magnetic field.

In further embodiments of the disclosure, a method of facilitating pulsatile blood flow includes positioning an assist device within a blood vessel of a patient. The device includes a drive shaft comprising a portion including piezoelectric or vibrational material. The portion of the device may be configured to move in response to an electric, electromagnetic, or magnetic field. The method additionally includes applying the electric, electromagnetic, or magnetic field to the portion of the device to change the movement (e.g., vibration) of one or more portions (e.g., balloons) of the device.

### BRIEF DESCRIPTION OF THE DRAWINGS

For a detailed understanding of the disclosure, reference should be made to the following detailed description, taken in conjunction with the accompanying drawings, in which like elements have generally been designated with like numerals, and wherein:

FIG. 1 illustrates a perspective view of a circulatory assist device and placement catheter, in accordance with embodiments of the disclosure.

FIGS. 2A and 2B illustrate perspective views of expanded and retracted positions of the circulatory assist device, in accordance with embodiments of the disclosure.

FIGS. 3A and 3B illustrate perspective views of expanded and retracted positions of an alternative embodiment of the circulatory assist device, in accordance with embodiments of the disclosure.

FIG. 4 illustrates a partial cross-sectional view of a placement catheter, in accordance with embodiments of the disclosure.

FIG. 5 illustrates a cross-sectional view of a balloon, in accordance with embodiments of the disclosure.

FIG. 6 illustrates a cross-sectional view of a blood vessel, in accordance with embodiments of the disclosure.

FIG. 7 illustrates a side view of a balloon and drive shaft, in accordance with embodiments of the disclosure.

FIG. 8 illustrates a schematic diagram of a sensor, in accordance with embodiments of the disclosure.

FIG. 9 illustrates a circulatory assist system, in accordance with embodiments of the disclosure.

FIG. 10 is a diagrammatic view of a patient, in accordance with embodiments of the disclosure.

FIG. 11 is an exploded view of a portion of the patient of FIG. 10.

FIG. 12 is a diagrammatic view of a portion of the patient, in accordance with embodiments of the disclosure.

FIG. 13 is a diagrammatic view of a portion of the patient, in accordance with embodiments of the disclosure.

FIG. 14 is a diagrammatic view of a portion of the patient, in accordance with embodiments of the disclosure.

FIG. 15 is a diagrammatic view of a portion of the patient, in accordance with embodiments of the disclosure.

### DETAILED DESCRIPTION

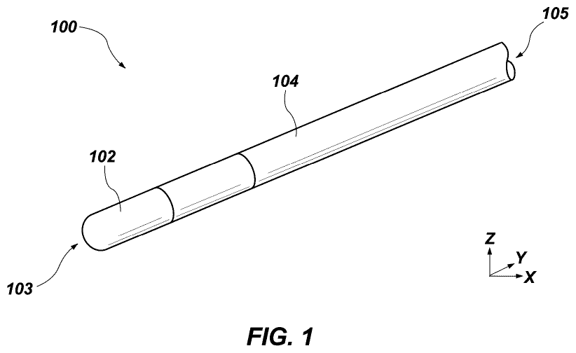

An aspect of the disclosure is a circulatory assist device, generally 100 and 101, shown in FIGS. 1, 2A, 2B, 3A and 3B in extended and retracted positions. As can be determined, the accompanying figure drawings are generally not drawn to scale.

The circulatory assist device may be placed, for example, in the aorta above the renal arteries to aid in kidney function or in the aorta to aid in heart function. More flow into the kidneys means more rapid removal of excess fluids, which leads to better revival of kidney function. In certain embodiments, the system preferably uses the full diameter of the renal arteries or the aorta to increase pump stability and reduce pump migration.

In certain embodiments, the circulatory assist device may be communicatively coupled with implanted sensors that assist with a real time, automatic adjustment and management of the circulatory assist device based upon data provided by the implanted (preferably wireless) sensors. The sensors monitor fluid flow and provide feedback and data to the circulatory assist devices, or a controller operatively coupled with the circulatory assist devices, which feedback and data is used to, e.g., adjust the speed and/or angle of the impeller, to increase or decrease fluid flow and pressure, or to increase or decrease piezoelectric vibration.

A wireless power embodiment is designed to reduce infection risk compared to external drive line systems. Also, the wireless power option helps improve a patient utilizing the device's quality of life. Typically, a patient would be a mammalian subject, such as a human.

Optionally, the circulatory assist device may be utilized with one or more cuff stent grafts, which improves the total flow, improves hemodynamics, (via the pulsatile flow) improves the release of beneficial proteins for organ health, and reduces RPMs needed by the impeller to reach desired flow rates.

In some embodiments, elements of the circulatory assist device as described herein (e.g., impeller blade(s), drive shaft, and/or stent cage) are coated with a hydrophobic or lubricous material to reduce the potential for endothelialization after placement of the circulatory assist device. Such a material can be, for example, expanded polytetrafluorethylene (ePTFE available from Gore Technologies) or similar graft liner.

Referring to FIG. 1, in some embodiments, the circulatory assist device 100 includes a distal end 103 and a proximal end 105. The distal end 103 of the device 100 includes a sensor 102 and the proximal end 105 includes a placement catheter 104. The placement catheter 104 may be sized and shaped for subcutaneous insertion within a patient 130 at an incision site 131 (FIG. 11, below).

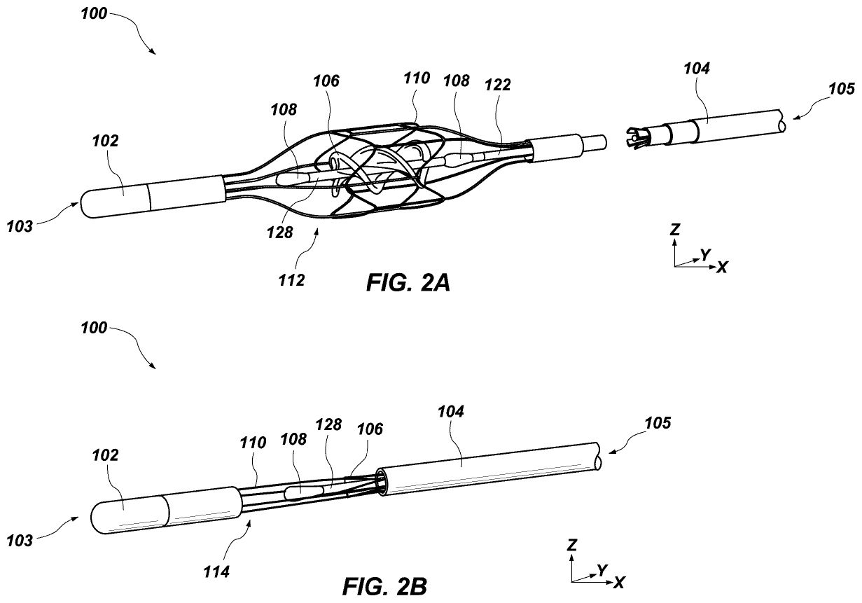

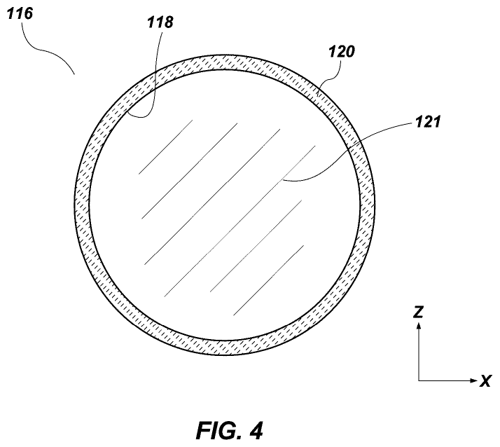

Referring to FIGS. 2A and 2B, the circulatory assist device 100 includes an impeller 106 positioned between the distal end 103 and the proximal end 105 thereof. In some embodiments, the impeller 106 includes a helical shaped, continuous (e.g., no intervening materials or holes) blade and a drive shaft 128 running along a center axis of the helical blade. In some of these embodiments, the helical blade of the impeller 106 includes holes, baffles, protrusions, or other materials, such that it may be semi-continuous or may have some discontinuity. A circumference of the helical blade may be slightly less than the inner circumference of a tubular elongated encasing (FIG. 4, below).

In some embodiments, the circulatory assist device 100 includes a sensor 102. In the embodiment illustrated, the sensor 102 is positioned (e.g., attached, without limitation) at the distal end 103. The sensor 102 is configured for receiving electromagnetic frequencies 146 for controlling the impeller 106. The impeller 106 is configured to be electrically activated (e.g., wirelessly or through direct electrical coupling) to move intravenous fluid 121 based upon the electromagnetic frequencies 146 received by the sensor 102.

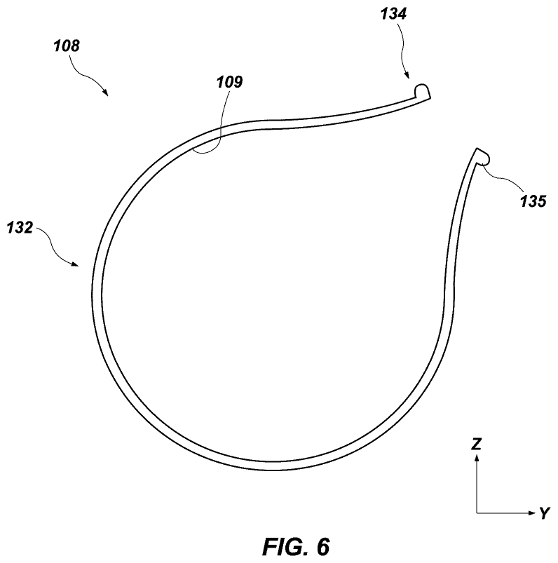

In some embodiments, the circulatory assist device 100 includes a balloon 108, the interior of which defines a balloon chamber (refer to FIG. 6). In some of these embodiments, two or more balloons 108 are connected coaxially and aligned with a drive line 122 and the drive shaft 128 of the impeller 106. The drive line 122 may be encased within a tubular elongated casing, which may extend from the impeller 106. In some embodiments, the two or more balloons 108, each including a balloon chamber (refer to FIG. 6) are positioned proximally and distally relative to the impeller 106. In other words, the impeller 106 is positioned between the two or more balloon chambers.

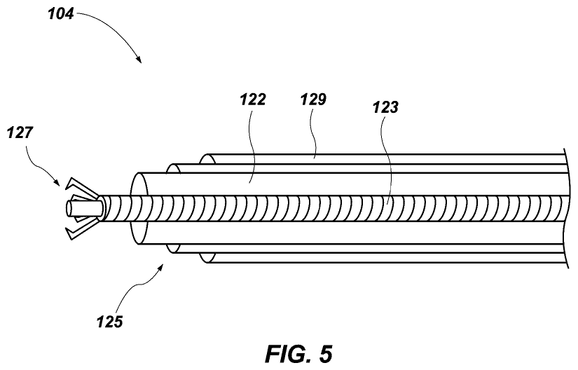

The circulatory assist device 100 includes a stent cage 110. The stent cage 110 is of a size and shape that allows it to be placed within a blood vessel 116 (FIG. 5, below). The stent cage 110 is configured to move between an expanded position 112 (FIG. 2A) and a retracted position 114 (FIG. 2B). In various embodiments, the stent cage 110 includes a highly open flow configuration, which may prevent damage to, e.g., the patient's blood cells, such as hemolysis. The highly open flow configuration may also reduce the risk of thrombosis. In some embodiments, the stent cage 110 includes one or more wires, each including surfaces smoothed or formed in a manner configured to reduce friction or damage to walls 120 of the blood vessel 116.

The stent cage 110 may be configured to be sufficiently rigid to maintain secure in the expanded (e.g., in the deployed or open state) position 112, braced against the blood vessel 116 (e.g., aorta) of the patient 130, while being sufficiently flexible to enable fluctuations due to natural pulsatility of the blood vessel(s) 116 of the patient 130.

Maintaining vessel wall motion during natural pulsatility may facilitate aortic protein expressions such as Klotho that promote multiple organ health especially kidney health and avoid plaque formation. Maintaining vessel wall motion during natural pulsatility may also improve blood pressure and hemodynamics. The benefits of natural pulsatility are discussed in the following article, the contents of which are incorporated herein by this reference: Why pulsatility still matters: a review of current knowledge, Davor Barić, Croatian Medical Journal, Volume 55(6), December 2014, pages 609-620, DOI: 10.3325/cmj.2014.55.609.

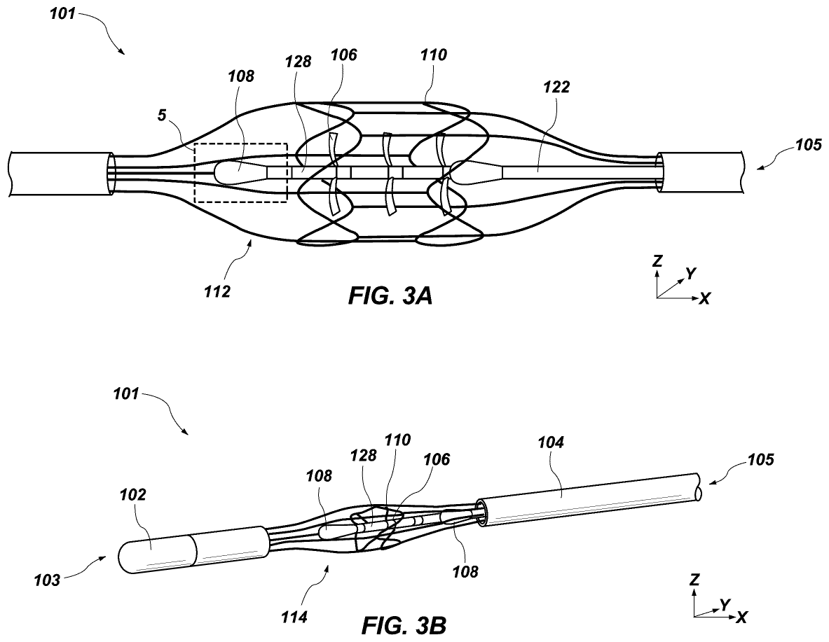

Referring to FIGS. 3A and 3B, in some embodiment, the impeller 106 includes one or more impeller blades configured to fold towards the drive shaft 128. In some of these embodiments, the depicted impeller blades are pivotally associated with a lobe by pivots (e.g., pins or shafts). The impeller blades are outwardly foldable and retractable, and can move, e.g., into a position perpendicular to the drive shaft 128. The impeller blades may be configured to fold concurrently with the stent cage 110 while the stent cage 110 is moved to the retracted position 114, while in other embodiments, the impeller blades may be actuated separately from the stent cage 110 (e.g., folded into drive shaft 128 while stent cage 110 is still in expanded position 112). In either embodiment, the stent cage 110 is configured to prevent contact between the wall 120 of the blood vessel 116 (e.g., the aortic tissue of the patient 130) and the impeller blades of the impeller 106.

In some embodiments, the impeller 106 includes a combination of the one or more impeller blades that are configured to fold and one or more helical shaped blades. The impeller 106 may include other types of impeller blades and may include any combination of impeller blades.

Referring to FIG. 4, in some embodiments, the retracted position 114 is circumferentially less than an inner circumference 118 of a wall 120 of the blood vessel 116, while the expanded position 112 is circumferentially greater than or equal to the inner circumference 118 of the wall 120 of the blood vessel 116.

Referring to FIG. 5, in some embodiments, the circulatory assist device 100/101 and the placement catheter 104 use a monorail guidewire lumen “rapid exchange” (“RX”) system, including the tubular elongated casing housing a guidewire lumen in the circulatory assist device 100/101. The portion of the RX system of the placement catheter 104 is configured to extend proximally a short distance from a tip thereof. See, e.g., US 2003/0171642 A1 to Schock et al. (Sep. 11, 2003) and J. Schroeder 2013 Peripheral Vascular Interventions: An Illustrated Manual, “Balloon Catheters Over the Wire and Monorail,” DOI: 10.1055/b-0034-65946, the contents of each of which are incorporated herein by this reference. In some embodiments, the placement catheter 104 includes a telescopic end 125 including a casing 133 telescopically integrated with one or more sleeves 129. For example, the casing 133 is surrounded by a sleeve 129 or a tube of an elastic material such as rubber or similar.

The placement catheter 104 includes a guidewire lumen 123 and a portion encased within the casing 133. In some embodiments, the guidewire lumen 123 is configured to connect with the guidewire lumen of the circulatory assist device 100 to supply a fluid thereto.

The placement catheter 104 includes a mechanism 127 configured to secure the circulatory assist device 100 to the placement catheter 104 for positioning the circulatory assist device 100 within the body of a patient. In some embodiments, the impeller 106 is configured to be wirelessly activated after the circulatory assist device 100/101 is detached from the mechanism 127. In some embodiments, the mechanism 127 is positioned at an end of the guidewire lumen 123.

In some embodiments, the drive line 122 and an “over the wire” (OTW) guidewire may be encased within a tubular elongated casing of the impeller 106, where the impeller 106 is directly electrically and mechanically coupled, and is actuated over a direct (e.g., wired) electrical connection. The impeller 106 is connected to the drive shaft 128 for actuating the impeller 106 upon receiving an activation signal.

Although the guidewire lumen 123 is depicted as a solid material, this depiction is for ease of illustration. It is important to note that in some embodiments, all, or at least some, of the guidewire lumen 123 is hollow, allowing a fluid (e.g., air) to pass to the guidewire lumen of the circulatory assist device 100/101 and into the balloons 108 of the circulatory assist device 100/101. In some embodiments, the guidewire lumen 123 is housed together with an actuation cable that may be used to actuate the circulatory assist device 100/101. In other embodiments, the actuation cable comprises the guidewire lumen 123, which may be substantially solid.

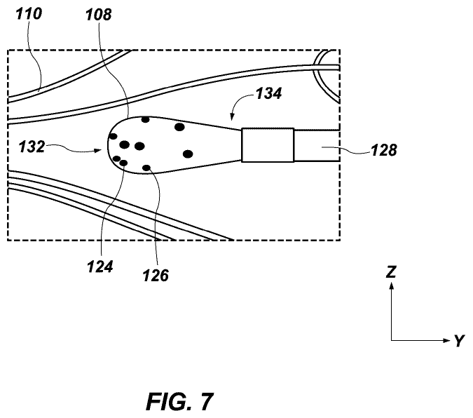

Referring to FIGS. 6 and 7, the balloon 108 includes a balloon wall 109 defining a chamber of the balloon 108. The balloon 108 includes a body portion 132 that smoothly or continuously transitions to an attachment portion 134. In other embodiments, the transition between the body portion 132 and the attachment portion 134 may be disjointed, due to fold lines as a result of the balloon 108 retracting while the circulatory assist device 100/101 is in the retracted position 114 prior to or after use of the balloon 108 and the circulatory assist device 100/101. The attachment portion 134 may be annularly shaped (e.g., circular annulus, square annulus, elliptical annulus, etc.), and may include a lip or ridge 135 configured to secure the balloon 108 to a portion of the drive shaft 128. While a single lip or ridge 135 is depicted, in some embodiments, the attachment portion 134 includes multiple lips or ridges 135. In some embodiments, the attachment portion 134 includes an adhesive or thermal bonding configured to attach the balloon to the drive shaft 128. In some of these embodiments, the attachment portion 134 includes one or more lips or ridges 135 and one or more of an adhesive and thermal bonding. In some embodiments, as illustrated in FIGS. 2A, 3A, and 3B, two balloons 128 are proximally and distally located at opposite ends of the drive shaft 128, the two balloons being separated by the impeller 106. The drive shaft 128, or a portion thereof, may be hollow for fluid communication (e.g., inflating and/or deflating) to the balloon chambers of the balloons 108 or may include a separate guidewire lumen therein.

The balloon wall 109 may include one or more materials. For example, a balloon wall 109 may include a piezoelectric material 124. The piezoelectric material 124 may comprise a piezoelectric crystal, such as perovskite crystals. The crystal structure may comprise a tetravalent metal ion in a lattice of large divalent metal ions. The piezoelectric crystal may comprise a variety of materials including one or more ceramics such as lead zirconate titanate (PbZrxTi1-xO3 with 0≤x≤1 (e.g., PZT-5A, PZT-5H, PZT 5-J, PZT-4, PZT-8)), potassium niobate (KNbO-3), sodium tungstate (Na2WO3), Ba2NaNb5O5, Pb2KNb5O15, zinc oxide (ZnO); lead-free piezoceramics such as sodium potassium niobate ((K,Na)NbO3), bismuth ferrite (BiFeO3), sodium niobate (NaNbO3), barium titanate (BaTiO3), bismuth titanate (Bi4Ti3012), sodium bismuth titanate (NaBi(TiO3)2); Group III-V and II-VI semiconductors such as gallium nitride (GaN), indium nitride (InN), aluminum nitride (AIN), zinc oxide (ZnO); polymers such as polyvinylidene fluoride (PVDF) and its copolymers, polyamides, parylene-C, polyimide, and polyvinylidene chloride (PVDC); and various other crystalline materials such as langasite (La3Ga5SiO14), gallium orthophosphate (GaPO4), lithium niobate (LiNbO3), lithium tantalate (LiTaO3), quartz, berlinite (AlPO4), rochelle salt, topaz, tourmaline-group minerals, and lead titanate (PbTiO3). The piezoelectric materials 124 may be organic piezoelectric biomaterials or inorganic materials. Organic piezoelectric biomaterials may include, but are not limited to, piezoelectric proteins, peptides, and other biopolymers. The piezoelectric materials 124 may be non-synthetic or synthetic materials. See, e.g., Shin, Dong-Myeong, et al., “Recent Advances in Organic Piezoelectric Biomaterials for Energy and Biomedical Applications,” Nanomaterials, Volume 10(1), Jan. 9, 2020, DOI 10.3390/nano10010123, the contents of which are incorporated by this reference in their entirety. Non-synthetic piezoelectric materials 124 may include, but are not limited to, Berlinite, cane sugar, quartz, Rochelle salt, topaz, tourmaline, bone, or combinations thereof. See, e.g., “The Piezoelectric Effect,” Nanomotion, a Johnson Electric Company, www.nanomotion.com/nanomotion-technology/piezoelectric-effect/(last visited Jun. 2, 2022).

The shape and size of the piezoelectric material 124 may vary depending on its desired movement and/or application. For example, relatively smaller spheroids, cuboids, granules, and such may be useful for mechanical vibrations; whereas, rods, cylinders, or relatively elongated structures may be useful for expansion and contraction movements.

The motions generated by the piezoelectric material 124 may simulate natural pulsatility and vessel wall movement. The piezoelectric material 124 may be configured to receive the electromagnetic frequencies 146 and move based upon the received electromagnetic frequencies 146. For example, in some embodiments, the piezoelectric material 124 may convert an electrical signal (e.g., wireless signal, radio frequency, etc.) into mechanical vibrations. In other embodiments, the piezoelectric material 124 may convert an electrical signal into a series of contractions and expansions (e.g., depending on shape and size of the piezoelectric material 124), providing an electro-mechanical simulation of natural, rhythmic blood flow.

Blood flow within the blood vessel 116 may be characterized by one or more fluid dynamic relationships, such as a Reynolds number, Bernoulli's Equation, and/or a Navier-Stokes equation. As the circumference of the blood vessel 116 decreases, e.g., due to plaque buildup, the preferred laminar flow of the intravenous fluid 121 may be converted to turbulent flow. See, e.g., Klabunde, Richard E., “Turbulent Flow,” Cardiovascular Physiology Concepts, Wolters Kluwer 2021, 3rd Ed. (www.cvphysiology.com/Hemodynamics/H007). Without being bound by theory, the vibrations of the piezoelectric material 124 may help to convert the turbulent flow back to laminar flow. For example, the vibrations may facilitate movements to vessel walls 120 that help increase the diameter of the walls 120; or, the vibrations may affect fluid velocity and/or flow rates, which also affect turbulent and laminar flows.

In additional embodiments, a vibration-enhancing material 126 may be contained within the balloon chambers, alone or in combination with the piezoelectric material 124. The vibration-enhancing material 126 may include a material (e.g., centrally symmetric) having atoms arranged in a lattice or a substantially uniform distribution, such as in metals, ceramics, and crystals. The vibration-enhancing material 126 may enhance vibrations from moving components of the circulatory assist device 100/101, such as the drive shaft 128.

The body portion 132, including the balloon wall 109, may comprise an elastomeric material such as, for example, silicone, and portions of the balloon wall 109 that define the balloon chamber may be slightly expandable, such that the balloon chamber can be inflated and vibrated after insertion of the circulatory assist device 100/101 into the body of the patient 130. Prior to insertion, the balloon chamber of each balloon 108 may be in a fully deflated state, allowing for low-profile insertion of the circulatory assist device 100/101 into the body of the patient 130. Once inserted, fluid (e.g., a gas, such as air, for example) may be caused to flow through guidewire lumen 123 of the placement catheter 104 to the driveshaft 128 of the impeller 106 and into the balloon chamber(s), facilitating the inflation and expansion for the desired vibration of the balloon(s) 108. In other embodiments, the balloon(s) 108 are formed of an elastomeric material that is compressible, such that the balloon(s) 108 are compressed when in a retracted state and decompressed when in an expanded state. In these embodiments, the balloon(s) 108 are not inflated through a lumen of the guidewire, but rather expand and contract due to the compression and decompression of the materials of the balloon and any compressible fluids (e.g., inert gas) contained therein.

The flexible, or elastomeric material of the body portion 132 of the balloon 108 is configured to vibrate based upon movement of the impeller 106. For example, the impeller 106 may increase, decrease, or comprise a wide range of blood flow rates during operation of the circulatory assist device 100/101 based upon a number of different factors (e.g., input from implantable sensors). The flexibility or elasticity of the material of the body portion 132 may be selected based upon an average anticipated flow rate, a threshold flow rate (e.g., max or min), or a target flow rate. Accordingly, with high average anticipated flow rates, materials having less elasticity or flexibility may be selected; conversely, with low average anticipated flow rates, materials having high elasticity or flexibility may be selected. Biocompatible rubbers, latexes, polymers (e.g., polypropylene), silicon, or combinations thereof may be among those selected.

In some embodiments, the body portion 132 generally comprises a tear drop shape. However, other shapes are also contemplated. For example, the body portion 132 may exhibit an annular shape, thereby allowing the drive shaft 128 to pass through a center portion of the annular shape thereof. Other shapes may include, but are not limited to, spherical, semi-spherical, elongated, and combinations thereof. The attachment portion 134 may comprise a ring shape, an annular shape, or combinations thereof, such that it may be inserted within, or placed around (e.g., over), an end of the drive shaft 128.

In some embodiments, two or more balloons 108 contain the piezoelectric material 124. In other embodiments, at least one balloon 108 contains the piezoelectric material 124, while at least another balloon 108 contains the vibration-enhancing material 126. In additional embodiments, the two or more balloons 108 each contain both the piezoelectric material 124 and the vibration-enhancing material 126. Multiple balloons 108 (e.g., three, four, five, or more) may be concentrically aligned with the drive shaft 128 of the circulatory assist device 100/101, and various material combinations within respective balloons 108 or groups of balloons 108 will be recognized and are included herein.

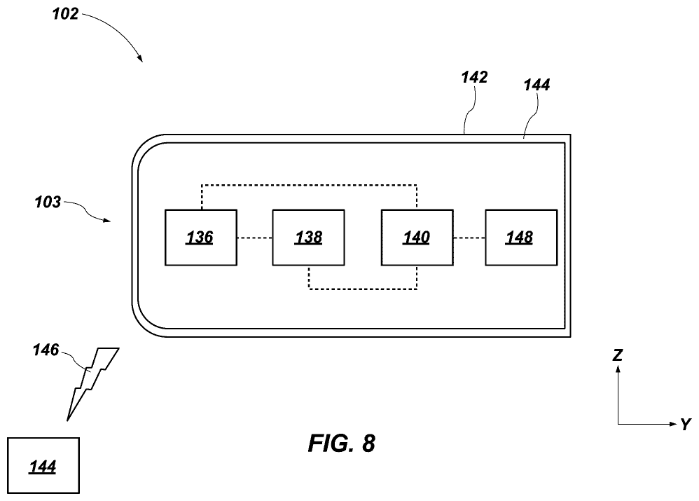

Referring to FIG. 8, in some embodiments, the sensor 102 includes a microprocessor 136, an electromagnetic (e.g., RF) receiver or transceiver 138, a power source 140 (e.g., battery), and a motor 148. The power source 140 may provide electrical power to the microprocessor 136, the transceiver 138, and the motor 148. The sensor 102 includes a body portion 142 (e.g., radome) that is made of a RF transparent material 144, such as TEFLON®, glass, plastics, and combinations thereof.

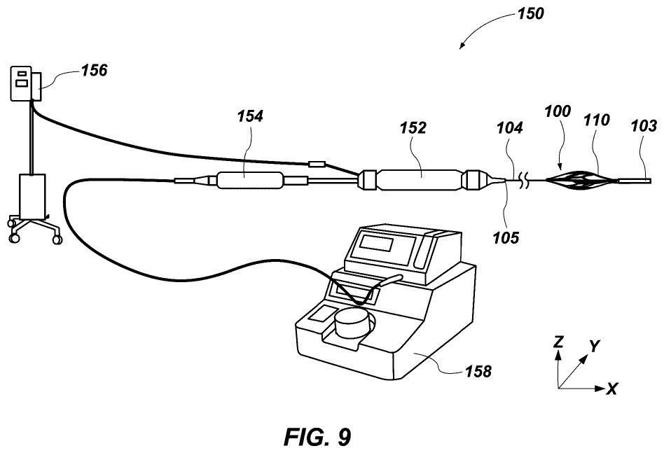

A transmitter 144 may transmit one or more electromagnetic signals (RF signals, Wi-Fi, BLUETOOTH®, etc.) 146 to the transceiver 138. Upon receipt of the electromagnetic signals 146, the microprocessor 136 may generate an activation signal which it sends to the motor 148. Upon receipt of the activation signal, the motor 148 then actuates the impeller 106. In other embodiments, one or more components of the sensor 102 may be removed and/or added. For example, one or more electromagnets may be included within the sensor 102. By way of another example, the motor 148 may not be present when the placement catheter 104 uses a direct electrical and/or mechanical connection a motor control drive unit 158 (FIG. 9, below); or additional flow sensors may be positioned distally at the tip of the sensor 102 to provide additional fluid flow feedback.

Referring to FIG. 9, a circulatory assist system 150 may include the placement catheter 104 and the circulatory assist device 100/101. The placement catheter 104 may be proximate the proximal end 105 of the circulatory assist system 150, and the circulatory assist device 100/101 may be proximate the distal end 103 of the circulatory assist system 150. The circulatory assist system 150 may include a first sheath driveline 152 that may be configured to rotate the impeller 106 (FIGS. 2A and 3A) of the circulatory assist device 100/101. The first sheath driveline 152 may be configured to control movement of the driveshaft 128 (FIG. 2A and 3A) and/or the placement catheter 104.

In some embodiments, the circulatory assist system 150 may include a second sheath driveline 154 that may be configured to facilitate movement and/or rotation of the casing 150 (FIGS. 2A and 3A) and/or the placement catheter 104. The circulatory assist system 150 may include a power supply 156 (e.g., medical grade UPS) that may facilitate transport and provide power to the circulatory assist system 150. Further, the circulatory assist system 150 may include a motor drive control unit 158 configured to facilitate movement and/or rotation of the impeller 106 (FIGS. 2A and 3A), the casing 122 (FIGS. 2A and 3A), and/or the placement catheter 104.

In some embodiments, sensors are used with the circulatory assist system 150, e.g., to monitor hemolysis and/or impeller speed, and the pulsations of cuffs are adjusted as desired to balance a minimization of hemolysis with a maximization of flow utilizing the system 150.

The circulatory assist system 150 may be used to not only sustain a (e.g., congestive heart failure) life of the patient 130, but also may be used to provide mechanical circulatory assistance for, e.g., up to 36 months, during the course of heart rehabilitation/regeneration treatment.

The impeller 106 (FIGS. 2A and 3A) of the circulatory assist device 100/101 and the circulatory assist system 150 may be configured to be operated (e.g., rotated) mechanically via the drive shaft 128 and an elongated portion of the placement catheter (FIG. 5) that may be operably coupled to the motor control drive unit 158. In some embodiments, the impeller 106 (FIGS. 2A and 3A) of the circulatory assist device 100/101 and the circulatory assist system 150 may be configured to be operated (e.g., rotated) wirelessly, as described in in U.S. 2021/0077687 A1 to Leonhardt and U.S. 2021/0008263 A1 to Leonhardt.

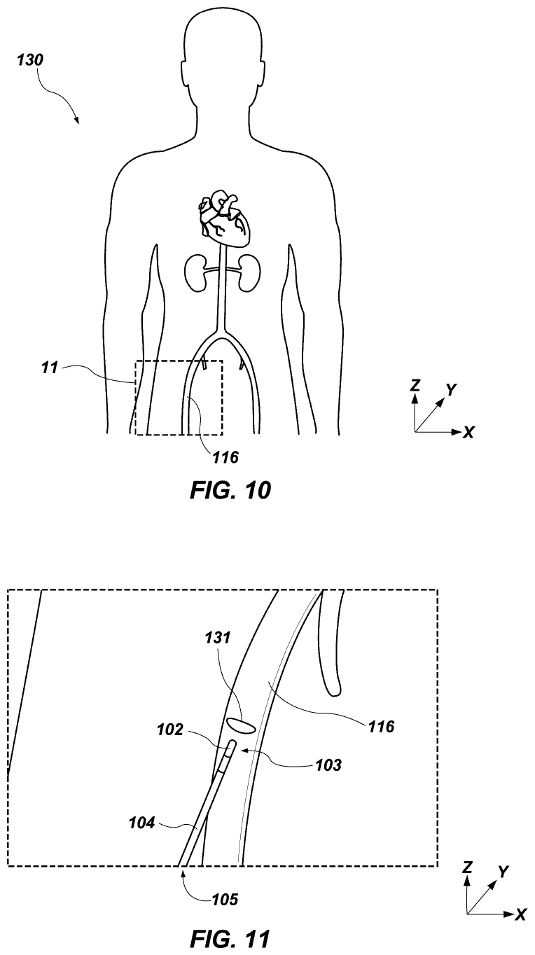

Referring to FIGS. 10 and 11, in operation, the patient 130 includes the blood vessel 116 (e.g., artery) that has been selected for an incision site 131. In FIG. 11, the dashed box circles the incision site 131 that is made more visible in the exploded view of FIG. 11.

The placement catheter 104 may be introduced “percutaneously” at the incision site 131 into the blood vessel 116 (e.g., lower aorta) via, e.g., the normal “Seldinger technique” in the groin (a small incision into the femoral artery) and fed up to the aorta to the desired position (e.g., the descending aorta). The circulatory assist device 100/101 may be inserted in the groin area and introduced into the blood vessel 116 (e.g., femoral artery to just above the renal arteries in the descending aorta) with the help of a small surgical insertion and insertion sheath. The circulatory assist device 100/101 is thereafter fed up into the desired position in the lower aorta.

Alternatively, the circulatory assist device 100/101 may be placed via axillary entry in the neck or chest of the subject. See, e.g., K M. Doersch “Temporary Left Ventricular Assist Device Through an Axillary Access is a Promising Approach to Improve Outcomes in Refractory Cardiogenic Shock Patients,” ASAIO J. 2015 May-June; 61(3): 253-258; doi: 10.1097/MAT.0000000000000222, the contents of which are incorporated herein by this reference in their entirety, which describes implantation of a temporary left ventricular assist device (“LVAD”) through an axillary approach as a way to provide adequate circulation to the patient, avoid multiple chest entries and infection risks.

In some embodiments, one or more cuff stent grafts (not shown) is used at the incision site 131 to improve the total flow of the circulatory assist device 100/101, improve hemodynamics, (via the pulsatile flow) improve the release of beneficial proteins for organ health, and reduce RPMs needed by the impeller to reach desired flow rates.

In some embodiments, the circulatory assist device 100/101 that is delivered at the incision site 131 includes the impeller 106 that is a wirelessly driven and contained within a high aortic force protective stent cage 110. The circulatory assist device 100/101 may be placed within an upper aorta pulsating aortic cuff stent graft in the patient 130.

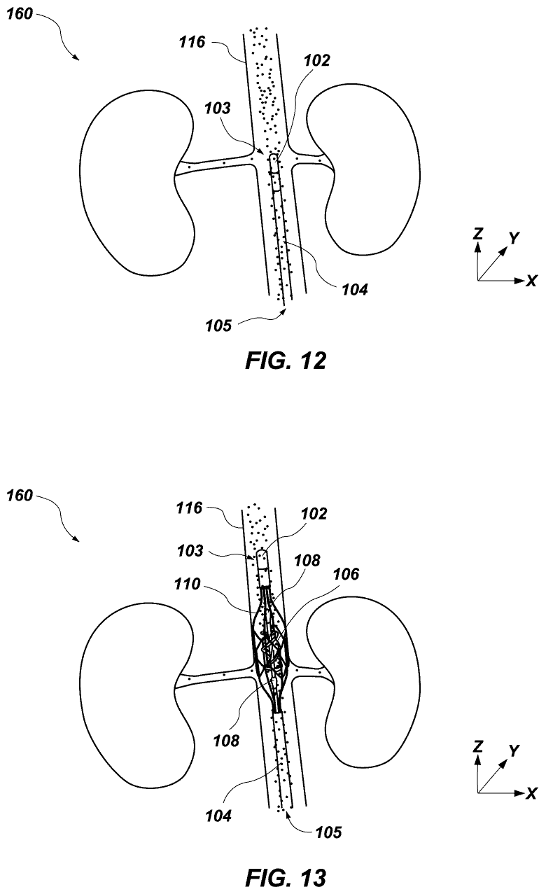

Referring to FIG. 12, the placement catheter 104 is used to position the circulatory assist device 100/101 including the sensor 102 at a desired intravenous site 160 within the patient 130. For example, the circulatory assist device 100/101 may be delivered just below the renal arteries that feed the kidneys while being held within an end of the placement catheter 104, such as within the sleeve 129 or the casing 133 of the placement catheter 104, while being secured thereto by the mechanism 127.

Referring to FIG. 13, upon reaching the intravenous site 160, the circulatory assist device 100/101 is withdrawn from the placement catheter 104. Upon being withdrawn from the placement catheter 104, such as from within the sleeve 129 or the casing 133, and being positioned within the blood vessel 116, the circulatory assist device 100/101 transitions into the expanded position 112 with the stent cage 110 expanding outward and contacting an inner wall of the blood vessel 116, which secures the circulatory assist device 100/101 in position. The transmitter 144 may be configured to transmit an electromagnetic frequency 146 (e.g., wireless signal or RF) to the transceiver 138 of the sensor 102, which may cause the impeller blades to expand.

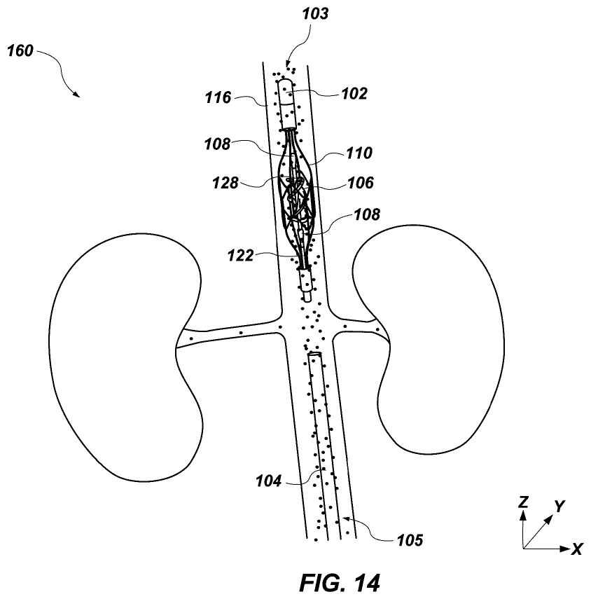

Referring to FIG. 14, the mechanism 127 is actuated to release the circulatory assist device 100/101 at the intravenous site 160. Upon release of the circulatory assist device 100/101, and/or upon receiving the first electromagnetic frequency 146 or a second electromagnetic frequency 146, the microprocessor 136 signals the motor 148 to actuate the impeller 106.

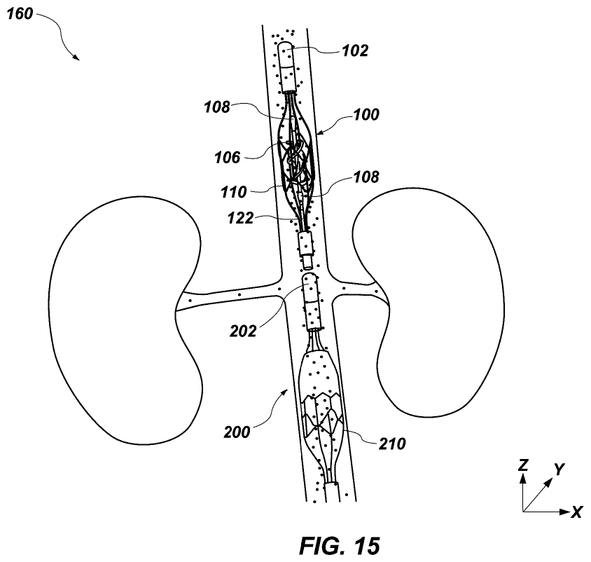

Referring to FIG. 15, although it is an advantage of the circulatory assist device 100/101 to not need to cross the renal arteries that feed the kidneys, in certain embodiments, the circulatory assist device 100/101 is included in a circulatory assist system that includes a second circulatory assist device 200 that is positioned on an opposing side of the renal arteries (e.g., in high head/low flow applications) relative to the circulatory assist device 100/101. Such a system includes placement of the circulatory assist device 100/101 positioned upstream of the renal valves at the tip of the placement catheter 104, beyond the renal arteries. In some embodiments, the second circulatory assist device 200 includes a sensor 202 (similar to sensor 102), a stent cage 210, and an impeller (not shown) proximal and or downstream of the renal arteries that feed the kidneys. The impeller, stent cage 210 and the sensor 202 may be similar to the features of the circulatory assist device 100/101. In some embodiments, one of the circulatory assist devices 100/101, 200is configured to extend the drive shaft further to interact and drive the other of the circulatory assist devices 100/101, 200. The two circulatory assist devices 100/101 and 200 are placed in series. In some embodiments, the two circulatory assist devices 100/101 and 200 are placed using the same catheter and may utilize the same drive shaft. In other embodiments, one or both of the two circulatory assist devices 100/101 and 200 are wirelessly actuated, such that they may or may not share the same drive shaft. In another embodiment, the second circulatory assist device 200 only includes a stent cage 210 and is configured to expand the blood vessel 116 and help ensure blood flow therethrough.

In additional embodiments, the circulatory assist device 100/101 may be placed to operate higher up in the patient 130 (e.g., at or near the heart, such as for left ventricle unloading). In these embodiments, the first circulatory assist device 100/101 is positioned past the heart valve and the second circulatory assist device 200 is positioned in the lower aorta, just above the renal arteries (for renal output improvement), i.e., the second circulatory assist device 200 is positioned in a blood vessel 116 at or adjacent to the mid to lower stomach and the first circulatory assist device 100/101 is positioned in the blood vessel 116 at or adjacent to the upper mid chest (usually 20 to 30 cm in most people). Treatment and/or PCI will typically continue for six (6) hours, but may last, for example, for 72 hours.

It is important to note that although the drive shaft and balloons containing piezoelectric or vibrational material is depicted relative to an inserted device, other configurations are contemplated and included herein. For example, the drive shaft, impeller, and balloons containing piezoelectric or vibrational materials may be used in an externally located left ventricular assist device. Other arrangements, configurations, and uses will be recognized by those skilled in the art and are included herein.

In the application above, the claims below, and in the accompanying drawings, reference is made to particular features (including method acts) of the disclosure. It is to be understood that the disclosure includes all possible combinations of such particular features. For example, where a particular feature is disclosed in the context of a particular embodiment, or a particular claim, that feature can also be used, to the extent possible, in combination with and/or in the context of other particular aspects and embodiments described herein.

The description above provides specific details, such as components, assembly, and materials in order to provide a thorough description of embodiments of the disclosure. However, a person of ordinary skill in the art will understand that the embodiments of the disclosure may be practiced without employing these specific details.

The use of the term “for example,” means that the related description is explanatory, and though the scope of the disclosure is intended to encompass the examples and legal equivalents, the use of such terms is not intended to limit the scope of an embodiment or this disclosure to the specified components, acts, features, functions, or the like.

Drawings presented herein are for illustrative purposes and are not necessarily meant to be actual views of any particular material, component, structure, or device. Thus, embodiments described herein are not to be construed as being limited to the particular shapes or regions as illustrated, but include deviations in shapes that result, for example, from manufacturing. For example, a region illustrated or described as box-shaped may have rough and/or nonlinear features, and a region illustrated or described as round may include some rough and/or linear features. Moreover, sharp angles that are illustrated may be rounded, and vice versa. Thus, the regions illustrated in the figures are schematic in nature, and their shapes are not intended to illustrate the precise shape of a region and do not limit the scope of the present claims. The drawings are not necessarily to scale. Additionally, elements common between figures may retain the same numerical designation.

As used herein, the term “configured” refers to a size, shape, material composition, material distribution, orientation, and arrangement of one or more of at least one structure and at least one apparatus facilitating operation of one or more of the structure and the apparatus in a predetermined way.

As used herein, the terms “comprising” and “including,” and grammatical equivalents thereof include both open-ended terms that do not exclude additional, unrecited elements or method acts, and more restrictive terms such as “consisting of” and “consisting essentially of” and grammatical equivalents thereof.

As used herein, the term “may” with respect to a material, structure, feature, or method act indicates that such is contemplated for use in implementation of an embodiment of the disclosure and such term is used in preference to the more restrictive term “is” so as to avoid any implication that other, compatible materials, structures, features and methods usable in combination therewith should or must be excluded.

As used herein, the singular forms “a,” “an,” and “the” include the plural forms as well, unless the context clearly indicates otherwise.

As used herein, the term “and/or” includes any and all combinations of one or more of the associated listed items.

As used herein, relational terms, such as “first,” “second,” etc., are used for clarity and convenience in understanding the disclosure and accompanying drawings and does not connote or depend on any specific preference, orientation, or order, except where the context clearly indicates otherwise.

As used herein, the term “about,” when used in reference to a numerical value for a particular parameter, is inclusive of the numerical value and a degree of variance from the numerical value that one of ordinary skill in the art would understand is within acceptable tolerances for the particular parameter. For example, “about,” in reference to a numerical value, may include additional numerical values within a range of from 90.0 percent to 110.0 percent of the numerical value, such as within a range of from 95.0 percent to 105.0 percent of the numerical value, within a range of from 97.5 percent to 102.5 percent of the numerical value, within a range of from 99.0 percent to 101.0 percent of the numerical value, within a range of from 99.5 percent to 100.5 percent of the numerical value, or within a range of from 99.9 percent to 100.1 percent of the numerical value.

As used herein, the term “substantially” in reference to a given parameter, property, or condition means and includes to a degree that one of ordinary skill in the art would understand that the given parameter, property, or condition is met with a degree of variance, such as within acceptable tolerances. By way of example, depending on the particular parameter, property, or condition that is substantially met, the parameter, property, or condition may be at least 90.0 percent met, at least 95.0 percent met, at least 99.0 percent met, at least 99.9 percent met, or even 100.0 percent met.

As used herein, the terms “biocompatible material” and “biocompatible materials” refer to any materials suitable for being within a subject's body. Biocompatible materials include ceramics and ceramic composites such as alumina (Al₂O₃), zirconia (ZrO₂), hydroxyapatite (Ca₁₀(PO₄)₆(OH)₂), and bioglass (e.g., composites including silica (SiO₂), calcium (Ca), sodium oxide (Na₂O), hydrogen (H), and/or phosphorous (P)). As non-limiting examples, bioglass may include 45S5 (e.g., 45% SiO₂, 24.5% CaO, 24.5% Na₂O, and 6% (P₂O₅)) and additional compositions described in the following article, the contents of which are incorporated herein by this reference: Vidya Krishnan and T. Lakshmi, “Bioglass: A novel biocompatible innovation,” Journal of Advanced Pharmaceutical Technology & Research, Volume 4(2), April-June 2013, pages 78-83, DOI: 10.4103/2231-4040.111523. Biocompatible materials may additionally include metals and metal alloys, such as stainless steel, titanium and titanium alloys (e.g., Nitinol), cobalt-chromium alloys (e.g., ASTM F75). Furthermore, biocompatible material may include polymers, such as polyvinylchloride (PVC), polyethylene (PE), polypropylene (PP), polytetrafluoroethylene (PTFE), expanded PTFE (ePTFE), polyetheretherketone (“PEEK”), Poly-paraphenylene terephthalamide (K29) (e.g., KEVLAR®), p-phenylene terephthalamide (PpPTA) (e.g., TWARON®), polymethylmethacrylate (PMMA), trimethylcarbonate (C₄H₆O₃), TMC NAD-lactide (CH₃[C₆H₈O₄]ₘ[C₄H₆O₃]ₙCH₃), polylactic acid (PLA), and medical-grade silicone.

As used herein, the term “blood vessel” means any vessel or artery used internally or externally relative to the body of a patient (e.g., humans or animals) that carries fluid, such as blood, to or from one or more organs of the patient.

As used herein, the term “piezoelectric” as used in reference to a material means any material (e.g., non-centrally symmetric) that is electrically tunable or experiences a contraction, elongation, or similar property change upon application, exposure to, or receipt of an electrical, magnetic, or electromagnetic field. The term may also refer to a material that generates an electric charge in response to applied mechanical stress.

### REFERENCES

(The contents of each of which are incorporated herein in their entirety by this reference)
U.S. Pat. No. 8,617,239, titled “Catheter Pump” to Reitan (Dec. 31, 2013).
U.S. Pat. No. 5,749,855, titled “Catheter Pump” to Reitan (May 12, 1998).
U.S. Pat. No. 4,753,221, titled “Blood Pumping Catheter and Method of Use” to Kensey et al. (Jun. 28, 1988).
Vidya Krishnan and T. Lakshmi, “Bioglass: A novel biocompatible innovation,” Journal of Advanced Pharmaceutical Technology & Research, Volume 4(2), Apr.-June 2013, pages 78-83, DOI: 10.4103/2231-4040.111523.
David Barić, “Why pulsatility still matters: a review of current knowledge,” Croatian Medical Journal, Volume 55(6), December 2014, pages 609-620, DOI: 10.3325/cmj.2014.55.609.
U.S. Patent App. Pub. No. 2003/0171642 A1, titled “Intra-aortic Balloon Catheter Having a Releasable Guide Wire” to Schock et al. (Sep. 11, 2003).
Juergen Schroeder, “Peripheral Vascular Interventions: An Illustrated Manual,” Thieme Verlagsgruppe, Stuttgart, NY, 2013, Balloon Catheters Over the Wire and Monorail, pages 27-28, DOI: 10.1055/b-0034-65946.
“What is ‘PZT’?,” APC International, LTD., www.americanpiezo.com/piezo-theory/pzt.html (last visited Jun. 2, 2022).
Dong-Myeong Shin, et al., “Recent Advances in Organic Piezoelectric Biomaterials for Energy and Biomedical Applications,” Nanomaterials, Volume 10(1), Jan. 9, 2020, DOI: 10.3390/nano10010123.
“The Piezoelectric Effect,” Nanomotion, a Johnson Electric Company, www.nanomotion.com/nanomotion-technology/piezoelectric-effect/(last visited Jun. 2, 2022).
Richard E. Klabunde, PhD, “Turbulent Flow,” Cardiovascular Physiology Concepts, Wolters Kluwer 2021, 3^{rd} Ed. (www.cvphysiology.com/Hemodynamics/H007).
U.S. Patent App. Pub. No. 2021/0077687 A1, titled “Circulatory Assist Pump” to Leonhardt.
U.S. Patent App. Pub. No. 2021/0008263 A1, titled “Circulatory Assist Pump” to Leonhardt.
U.S. Pat. No. 11,602,627 B2 (Mar. 14, 2023), titled “Circulatory Assist Pump” to Leonhardt.
K M. Doersch “Temporary Left Ventricular Assist Device Through an Axillary Access is a Promising Approach to Improve Outcomes in Refractory Cardiogenic Shock Patients,” ASAIO J., 2015 May-Jun; 61 (3): 253-258; DOI: 10.1097/MAT.0000000000000222.

What is claimed is:

1. A circulatory assist device comprising:
a distal end and a proximal end;
an impeller;
a sensor attached at least one of the distal and proximal ends configured to receive electromagnetic frequencies for controlling the impeller, the impeller configured to move fluid in response to received electromagnetic frequencies; and
two or more chambers positioned proximally and distally relative to the impeller, the impeller being positioned between the two or more chambers.

2. The circulatory assist device of claim 1, wherein the two or more chambers include vibrational or piezoelectric materials positioned in walls therein and configured to move or vibrate in response to receiving one or more of the electromagnetic frequencies.

3. The circulatory assist device of claim 1, further comprising a stent cage of a size and shape to be placed within a subject's blood vessel, the stent cage configured to expand within the subject's blood vessel.

4. The circulatory assist device of claim 3, wherein the stent cage is configured to transition between an expanded position and a retracted position.

5. The circulatory assist device of claim 4, wherein the retracted position is circumferentially less than an inner circumference of a wall of the blood vessel, and the expanded position is circumferentially greater than or equal to the inner circumference of the wall of the blood vessel.

6. The circulatory assist device of claim 1, wherein the two or more chambers are positioned coaxially along a drive line of the impeller and connected thereto.

7. The circulatory assist device of claim 1, wherein the two or more chambers contain a piezoelectric material positioned within the chambers.

8. The circulatory assist device of claim 7, wherein the piezoelectric material is configured to receive the electromagnetic frequencies and move based upon the received electromagnetic frequencies.

9. The circulatory assist device of claim 1, wherein the two or more chambers include a vibrational material positioned therein.

10. The circulatory assist device of claim 9, wherein the vibrational material enhances vibrations from a drive shaft.

11. The circulatory assist device of claim 10, wherein the two or more chambers comprise balloons configured to expand in response to a fluid being supplied thereto.

12. A circulatory assist device for moving fluid through a subject's vein or artery, the device comprising:
a stent cage of a size and shape to be placed within an artery or vein of a subject;
an impeller, positioned within the stent cage and configured to move fluid; and
two or more chambers positioned proximally and distally relative to the impeller, the impeller being situated between the two or more chambers.

13. The circulatory assist device of claim 12, wherein the impeller comprises a helical impeller.

14. The circulatory assist device of claim 12, further comprising:
a sensor for receiving electromagnetic frequencies for controlling the impeller.

15. The circulatory assist device of claim 14, wherein the impeller is wirelessly connected to the sensor is configured to control the impeller in response to the received electromagnetic frequencies.

16. The circulatory assist device of claim 12, wherein the two or more chambers each comprise a balloon comprising a flexible material configured to vibrate based at least in part upon movement of the impeller.

17. The circulatory assist device of claim 16, wherein the flexible material is annularly shaped for passing a driveshaft therethrough.

18. The circulatory assist device of claim 16, further comprising piezoelectric material positioned within the two or more chambers.

19. The circulatory assist device of claim 12, wherein each of the two chambers comprises a body portion and an attachment portion, the body portion comprising a tear drop shape and the attachment portion comprising an annular shape.

20. A circulatory assist device for placement within a blood vessel of a patient, the circulatory assist device comprising:
an impeller; and
at least one portion of piezoelectric and/or vibrational material, the piezoelectric and/or vibrational material configured to vibrate or resonate in response to application of an electric, electromagnetic, and/or magnetic field.
